(19) United States
(12) Patent Application Publication
(10) Pub. No.: US 2026/0130607 A1
(43) Pub. Date: May 14, 2026

(54) A HEARING ESTIMATION SYSTEM

(71) Applicant: Widex A/S, Lynge (DK)

(72) Inventors: Rasmus Malik Hoeegh LINDRUP, Lynge (DK); Jens Brehm Bagger NIELSEN, Lynge (DK); Lasse Lohilahti MOELGAARD, Lynge (DK); Caspar Aleksander Bang JESPERSEN, Lynge (DK)

(73) Assignee: Widex A/S, Lynge (DK)

(21) Appl. No.: 19/119,275

(22) PCT Filed: Oct. 9, 2023
(86) PCT No.: PCT/EP2023/077937

§ 371 Date: Apr. 8, 2025

(30) Foreign Application Priority Data
Oct. 10, 2022 (DK) PA202200921

(51) Int. Cl. A61B 5/12; A61B 5/00; G06N 3/0455; G06N 3/088

(52) U.S. Cl. CPC A61B 5/123; A61B 5/7267; A61B 5/7275; A61B 5/7475; G06N 3/0455; G06N 3/088

(57) ABSTRACT

A hearing estimation system (100) adapted to use a latent representation to provide a predictive distribution of a complete audiogram for a specific person.

The present invention relates to a hearing estimation system.

### BACKGROUND OF THE INVENTION

In a traditional hearing aid fitting, the hearing aid user goes to a site of a hearing aid fitter (e.g., an acoustician), and the user's hearing aids are adjusted using the fitting equipment that the hearing aid fitter has in his office. Typically, the fitting equipment comprises a computer capable of executing the relevant hearing aid programming software and a programming device adapted to provide a link between the computer and the hearing aid.

Traditionally a hearing aid system is fitted—initially or as part of a subsequent fine tuning-based primarily on a recorded audiogram for the hearing impaired person.

Thus an audiogram is a graphical representation of an individual's audible thresholds as a function of frequency. Traditionally, an audiogram is measured at a given set of frequencies, and taken together, these thresholds jointly characterize the hearing loss, or lack thereof, of a person. Beyond its diagnostic purpose, the audiogram is also used prescriptively to treat an individual's hearing loss, e.g. by defining frequency-specific gains in a hearing aid to compensate for the loss of audibility.

Perhaps the most widespread method is based on pure tone tests, where the person to be tested (i.e. the test person) is presented for a tone at a specific frequency and at first at a very low loudness that most probably is not audible for the test person, where after the loudness is progressively increased until the test person indicates that the tone is audible whereby the hearing threshold may be established, and from that the hearing loss at that specific frequency as compared to normal hearing subjects may be derived. In order to fully characterize the hearing loss, the test is repeated for other frequencies in the audible range. Obviously, this approach can be varied in a multitude of ways e.g. by first presenting a tone at a specific frequency with a very high loudness and then decreasing the loudness progressively.

This type of test has been offered as online test for many years. Hereby, a person who suspects possibly having a hearing loss can take the test at home and record their audiogram without having to make an appointment and travel to a hearing care professional. However, this type of test may be time consuming and some users consider the test uncomfortable and annoying, which additionally may lead to a recorded audiogram of low accuracy.

Thus measuring a complete audiogram is a time-consuming process, and in practice, experienced clinicians tend to rely on their domain knowledge to hasten the process so that they can determine when it is acceptable and appropriate to measure, for example, only a specific subset of frequencies. Obviously it is difficult to provide an automated (e.g. online) audiogram test capable of doing the same as the experienced clinician.

It is therefore an object of the present invention to provide a hearing estimation system adapted to provide an automated audiogram test that is both accurate and time-efficient.

More specifically it is an object of the present invention to provide audiogram acquisition that is fast, while also being accurate and enable this to be achieved with less experienced clinicians, or by fully automated systems.

In other words, it is also an object of the present invention to estimate a complete audiogram based on as few measured frequency dependent hearing thresholds as possible.

### SUMMARY OF THE INVENTION

According to a first aspect of the invention, an improved hearing estimation system for estimating an audiogram for a specific user is given according to claim 1.

### BRIEF DESCRIPTION OF THE DRAWINGS

The attributes and properties as well as the advantages of the invention which have been described above are now illustrated with help of drawings of an embodiment example. In detail,

FIG. 1 illustrates highly schematically a hearing estimation system according to an embodiment of the invention.

### DETAILED DESCRIPTION OF THE INVENTION

Initially it is noted that in order to facilitate reading of the description it may not always be explicitly mentioned that some data set or vector comprising frequency dependent hearing thresholds also comprises meta data. In this respect it is noted that meta data if available in most (if not all) cases will be acquired and thus be known from the beginning and consequently that any aspects directed at how to acquire new data will mainly (if not only) be directed at additional frequency dependent hearing thresholds.

But it is emphasized that generally meta data and frequency dependent hearing threshold data are treated in completely the same manner, with respect to the algorithms, which may also be denoted use cases, that are described in the following.

According to an embodiment of the present invention a variational autoencoder (that in the following may be abbreviated a VAE) has been trained to learn a representation (that in the following may be denoted a latent representation or a latent space) that can be used to characterize and predict a persons frequency dependent hearing loss in the form of a plurality of frequency dependent hearing thresholds for each ear.

Generally, VAEs learn representations by jointly optimizing an encoder and a decoder network, wherein the encoder maps data to a latent space and wherein the decoder learns to map from the latent space back to the original data space. Thus according to an embodiment the VAE has been trained by optimizing the evidence lower bound (which in the following may be abbreviated ELBO), which amounts to minimizing the distortions introduced by the composition of the encoder and decoder function under a constraint on the rate of information passed through the latent space.

More specifically the VAE can be trained to provide a latent representation that provides a trade-off between characterizing the observed data well (quantified as a negative log-likelihood, or distortion) while keeping the latent representation well-behaved against some predefined prior (quantified as a Kullback-Leibler divergence (KL), or rate). In other words, there exists a tension between having a “well-behaved” latent representation with a low rate and a model that captures as much as possible about the data with low distortion.

The ELBO can provide a balance that directly optimizes the log-marginal likelihood on the observed data.

However, alternative objectives for optimization exist, e.g. some that penalize the rate to a lesser or greater extent and hereby may improve the properties of the learnt representation in some aspects.

According to one specific embodiment the hearing estimation system therefore comprises at least two variational autoencoders, wherein one is optimized for predicting the complete audiogram and wherein the at least one other variational autoencoder is trained for optimized performance with respect to one of:
selecting the next frequency for which to measure a frequency dependent hearing threshold; and
selecting the next sound pressure level to use for initiating the determination of a frequency dependent hearing threshold, and
estimating the uncertainty of an estimated complete audiogram, and
determining when to stop acquiring more frequency dependent hearing thresholds.

In other words, the VAE is trained in order to enable estimation of a complete audiogram from as few measured frequency dependent hearing thresholds as possible. Therefore a model is defined that enables a determination of which frequencies to measure (a hearing threshold for) in an informed, sequential manner so as to arrive at a sufficiently accurate estimate with as few measurements as possible. Such a model can be said to have a good estimation performance. The number of measurements needed for a model with good estimation performance will, however, be directly dependent on the desired accuracy. Furthermore, the number of measurements, and at which frequencies, will vary from individual to individual. Therefore a model that is capable of quantifying the uncertainty of its estimate for a specific individual as the acquisition is in progress is desired, in order to determine at which point the process can be stopped. A model that achieves this is in the following said to have good uncertainty quantification.

Thus according to the present invention the VAE has been trained in order to provide a representation of audiogram data that enable optimization of (acquisition) estimation performance and uncertainty quantification as a function of rate-distortion trade-offs.

More specifically the VAE has been trained based on rate-dependent qualities of the representation in order to enable optimization of (i) the efficiency and accuracy of estimating complete audiograms from partially observed audiograms, and (ii) the ability to quantify the uncertainty of the estimation.

Thus, the training of the VAE has involved defining a partially observed audiogram xₒ (which in the following may also be denoted incomplete audiogram), which is a vector that has a set of observed dimensions O and unobserved dimensions U, wherein said dimensions jointly correspond to the dimension of a fully observed audiogram x, (which in the following may also be denoted the fully observed data or the complete audiogram). It is noted that in the present context the term audiogram may also represent so called meta data such as age in addition to a number of frequency dependent hearing thresholds for at least one of the hearing impaired persons two ears.

Now given a partially observed audiogram xₒ the inference network (which in the following may also be denoted the encoder part of the VAE) initially embeds each observed dimension, x_{d}. The embedding is aggregated across the observed dimensions and the aggregated embedding is fed to a network that parametrizes an approximate posterior distribution q_{φ} (z|xₒ) over a latent variable, z, conditioned on the partially observed audiogram xₒ, where φ are the collected parameters of the inference network.

The generative network produces distributions p_{θ} (x|z) of the fully observed data, x, given the latent variable z. If now assuming the observed and unobserved dimensions are conditionally independent given the latent variables, we get:

$${p}_{θ} ( x \vert z ) = {\prod }_{u ∈ U} {p}_{θ} ( {x}_{u} \vert z ) {\prod }_{o ∈ O} {p}_{θ} ( {x}_{o} \vert z ) \qquad ( 1 )$$

Where θ are the parameters of the generative network.

We follow common practice and use Gaussians for the observation distribution.

We optimize the inference and generative networks jointly by optimizing a lower bound on the log-marginal likelihood on the observed data, which in the following may be denoted the partial ELBO or Lₚ:

$$Log {p}_{θ} ( {x}_{o} ) \geq {L}_{p} = {E}_{z - {q}_{φ} ( z \vert {x}_{o} )} [ {\sum }_{o ∈ O} Log {p}_{θ} ( {x}_{o} \vert z ) - {D}_{KL} ( {q}_{φ} ( z \vert {x}_{o} ) \vert \vert p ( z ) ) ] = - ( {D}^{p} + R ) \qquad ( 2 )$$

where D^{p}, denotes the expectation with respect to the approximate posterior E_{z˜q}_{_{φ}} (z|xₒ) of the negative log-likelihood of the observed data Log p_{θ} (xₒ|z), and
where R denotes the expectation with respect to the approximate posterior E_{z˜q}_{_{φ}} (z|xₒ) of the Kullback-Leibler divergence D_{KL} (q_{φ} (z|xₒ)∥p(z)) from the prior p(z) and wherein the prior p(z) is a multi-variate normal distribution of the latent variable z, such as a standard isotropic Gaussian,
wherein xₒ is a vector comprising at least one of an observed frequency dependent hearing threshold and a meta data for said specific person. In other words xₒ is a vector comprising at least one observed dimension, and
wherein xₒ represents an observed dimension of xₒ.

Next the trained VAE is used to acquire a predictive distribution of a complete audiogram for a specific person sequentially by using the approximate posterior given the partial observation at any given time in the process to estimate the unobserved dimension.

In the following the number of measurements (which may also be denoted observations or dimensions) will be denoted by m and M will represent the total number of frequencies to be measured in order to obtain a complete audiogram. It is noted that as already discussed above the complete audiogram will typically comprise at least one additional dimension (in the form of so called meta data).

Now, the distribution over the unobserved dimensions, xᵤ, given a partially observed audiogram xₒ, may be determined as:

$${p}_{θ} ( {x}_{u} \vert {x}_{o} ) = {p}_{θ} ( {x}_{u} \vert z ) {p}_{θ} ( z \vert {x}_{o} ) \approx {p}_{θ} ( {x}_{u} \vert z ) {q}_{φ} ( z \vert {x}_{o} ) \qquad ( 3 )$$

Thus, an estimate of a complete audiogram can be obtained by combining the known xₒ with the mean of the predictive distribution given in equation (3) for each unobserved dimension, xᵤ

According to an embodiment the hearing estimation system (more specifically the graphical user interface) is configured such that the first acquisition is always the age of the specific person, because it has been shown that from this data alone a reasonable estimate of the predictive distribution can be obtained.

In other words an estimate of a complete audiogram based on an only partially observed audiogram can be obtained by first determining the predictive distribution p(x|xₒ) that is given by:

$$p ( x \vert {x}_{o} ) = \int p ( {x}_{u} \vert z ) p ( {x}_{o} \vert z ) p ( z ) d ( z ) , \qquad ( 4 )$$

and determining the average uₓ of the predictive distribution as:

$${μ}_{x} = {E}_{p ( x \vert {x}_{o} )} [ x ] = \int x p ( x \vert {x}_{o} ) dx , \qquad ( 5 )$$

and using μₓ as the estimate of the complete audiogram,
wherein as already given above x represents the complete audiogram, z is the latent variable and xᵤ represents unobserved frequency dependent hearing thresholds and wherein xₒ represents observed frequency dependent hearing thresholds.

Now in order to determine the next dimension (i.e. frequency) i∈U for which a next frequency dependent hearing threshold is to be acquired, an acquisition function R(u, xₒ), u∈U, based on the predictive distribution variance can be determined.

More specifically the next frequency for which to measure a frequency dependent hearing threshold can be determined based on the equation:

$$i = {arg max}_{u ∈ U} R ( u , {x}_{o} ) = {arg max}_{u ∈ U} {E}_{z - {q}_{φ} ( z \vert {x}_{0} )} [ Var ( {p}_{θ} ( {x}_{u} \vert z ) ) ] , \qquad ( 6 )$$

wherein i represents the next frequency to select,
wherein E_{z˜q}_{_{φ}} (z|xₒ) is the expectation with respect to the approximate posterior of the variance of the posterior predictive of the unobserved dimensions p_{θ} (xᵤ|z)
wherein Var(p_{θ} (xᵤ|z)) is approximated using the sample variance of samples from the posterior predictive of the unobserved dimensions p_{θ} (xᵤ|z) given multiple samples from the approximate posterior q_{φ} (z|xₒ).

According to another aspect of the present invention the next sound pressure level to use for initiating the determination of the next frequency dependent hearing threshold can be determined directly from the average of the predictive distribution at said next frequency. Hereby a significant reduction of the number of test tones the specific user needs to listen too can be achieved.

However, the learnt representation can also be used to estimate the uncertainty Q of an estimated complete audiogram, which can be determined from the equation:

$$Q = {\sum }_{i = 1}^{M} Var ( {x}_{i} ) = {\sum }_{i = 1}^{M} {[ Var ( x ) ]}_{i} = {\sum }_{i ∈ U} {E}_{z - {q}_{φ} ( z \vert {x}_{0} )} ( {p}_{θ} ( {x}_{u} \vert z ) ) \qquad ( 7 )$$

wherein M is the total number of frequencies to be measured in order to obtain a complete audiogram,
wherein E_{z˜q}_{_{φ}} (z|xₒ) is the expectation with respect to the approximate posterior of the variance of the posterior predictive of the unobserved dimensions p_{θ} (xᵤ|z).

Thus by introducing an uncertainty threshold, a simple method for determining when to stop the acquisition process (because the estimated complete audiogram is sufficiently accurate) can be obtained by simply detecting when the estimated uncertainty Q drops below the uncertainty threshold.

However, According to an alternative approach a method of determining when to stop the acquisition process is based on using a model to predict when to stop acquiring more frequency dependent hearing thresholds based on the predicted error of the estimate of the complete audiogram.

The model can be selected from a group of models comprising neural networks, linear models or non-linear models, such as least square models.

Preferably the models are trained using ground truth data from real audiogram acquisitions, to provide supervised training of the model using as input to the model at least the (current) number of measured frequency dependent hearing thresholds and a (current) estimated uncertainty of an estimated complete audiogram.

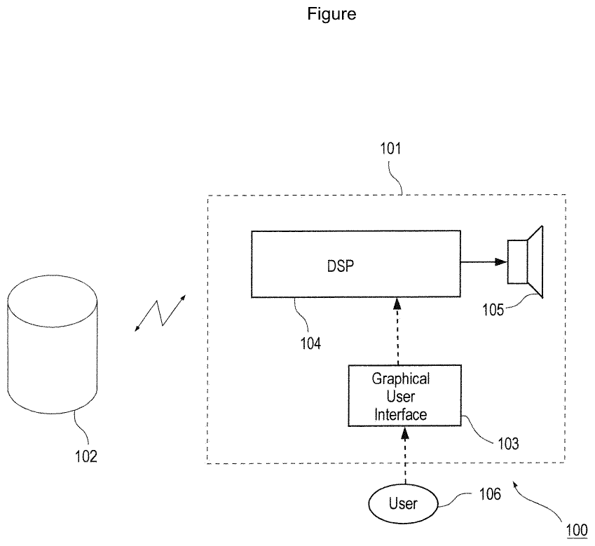

Reference is now made to FIG. 1, which illustrates highly schematically a hearing estimation system 100 according to an embodiment of the invention. The hearing estimation system 100 comprises a computerized device 101 and an external server 102.

The computerized device 101 further comprises a graphical user interface 103, a digital signal processor (DSP) 104 and an electro-acoustical transducer 105.

According to more specific embodiments the computerized device 101 may be a smart phone, a tablet computer, a portable personal computer or a stationary personal computer.

The external server 102 comprises a model (not shown) that has been trained to learn a latent representation of a plurality of audiograms and associated meta data, wherein said plurality of audiograms and associated meta are provided from a plurality of hearing impaired persons wherein each of said hearing impaired persons has provided at least one of a complete or incomplete audiogram, and at least one associated meta data.

Furthermore said model, comprising said latent representation (e.g. in the form of a variational autoencoder) is adapted to provide at least one of:
estimating a complete audiogram based on an average of the predictive distribution, and
selecting the next frequency for which to measure a frequency dependent hearing threshold; and
selecting the next sound pressure level to use for initiating the determination of a frequency dependent hearing threshold, and
estimating the uncertainty of an estimated complete audiogram, and
determining when to stop acquiring more frequency dependent hearing thresholds, wherein all of the above is based on receiving from the computerized device 101 at least one of an incomplete audiogram of said specific person, and at least one meta data of said specific person.

Thus, both the computerized device 101 and the external server 102 comprises a wireless link (not shown) adapted to transmit data, such as those described above in the paragraph above, in both directions between the computerized device 101 and the external server 102. More specifically, this functionality is provided using an application programming interface (API), such as a web service, that enable e.g. a web browser or a mobile application (i.e. an “app”) in the computerized device 101 to access and interact with the external server 102.

The graphical user interface 103 is adapted to enable a specific person 106 (which in the following may also be denoted a user) to provide at least one of an incomplete audiogram (which may consist of a single measured frequency dependent hearing threshold) and at least one meta data of said specific person to the hearing estimation system 100.

According to one embodiment said meta data comprises the user's age and the hearing estimation system 100 is configured to initially ask for and receive—through the graphical user interface 103—the age of the user wherefrom an initial predictive distribution of a complete audiogram for the user can be provided by transmitting the age to the server 102.

According to one embodiment said incomplete audiogram consist of at least one frequency dependent hearing threshold that has been obtained using the electro-acoustical transducer 105 to provide test sounds in reponse to input from the user (typically whether the test sound is audible or not) through the graphical user interface 103 and under control of the DSP 104 until at least one frequency dependent hearing threshold has been obtained using methodology that is well know within the field of audiometry.

The electro-acoustical transducer 105 is normally part of a set of standard headphones or earphones connected to the computerized device which enables an acoustical test signal that is selectively provided to either the left ear or the right ear.

According to an alternative embodiment the above mentioned model, comprising said latent representation (e.g. in the form of a variational autoencoder) is stored in the computerized device 101 instead of in the external sever 102, whereby the user will experience an even faster response time and consequently that the time required to obtain a complete audiogram or an estimated complete audiogram of sufficient precision can be minimized.

Thus according to this alternative embodiment an external server 102 will still be part of the hearing estimation system 100, but only to carry out the training of the above mentioned and later transfer the trained model to the computerized device.

According to yet another alternative embodiment the computerized device 101 and the external server may be integrated in one single device such as the personal computer of a hearing care professional, whereby the hearing care professional can train the model (e.g. in the form of an autoencoder or some other neural network) based on available data from hearing impaired users.

Thus according to different embodiments the latent representation is provided by an autoencoder such as a variational autoencoder or a partial variational autoencoder.

However, e.g. principal component analysis (PCA) can also be used instead of at least one of the encoder or decoder neural network of an autoencoder.

According to an embodiment p(x|z) is not parameterized, instead a function q(x|z) is parameterized, which is an approximation of p(x|z).

However, other encoder and decoder parameterizations may be implemented instead of this specific embodiment.

It is generally noted that even though many features of the present invention are disclosed in embodiments comprising other features then this does not imply that these features by necessity need to be combined.

As one example a number of various use cases derive from having a hearing estimation system adapted to provide a learnt latent representation based on having for each of a plurality of hearing impaired persons at least one of: a complete or incomplete audiogram, and at least one meta data and based hereon provide a predictive distribution of a complete audiogram for a specific person, based on at least one of: an incomplete audiogram of said specific person, and at least one meta data of said specific person.

However, these various use cases are generally independent, which means that e.g. the inventive feature of providing a predictive distribution of a complete audiogram for a specific person can be used to provide at least one of:
estimating a complete audiogram based on an average of the predictive distribution, and
selecting the next frequency for which to measure a frequency dependent hearing threshold; and
selecting the next sound pressure level to use for initiating the determination of a frequency dependent hearing threshold, and
estimating the uncertainty of an estimated complete audiogram, and
determining when to stop acquiring more frequency dependent hearing thresholds.

Thus according to one specific embodiment all of these use cases are based on the specific method of training a variational autoencoder to provide the latent representation, but according to other specific embodiments only one or two of the use cases (and these can be freely selected from the original three) are based on said specific method of training.

In a similar manner the feature of using a partial variational autoencoder can be combined with the various use cases independent on the number of use cases.

Overall any features related to a specific implementation of one of the different use cases may be combined with any of the specific implementations directed at learning the latent representation. In particular the specific types of neural networks that may be used to provide the latent representation.

Additional both of the above mentioned specific implementations may be combined with any of the specific methods of training a neural network to provide a latent representation, such as whether to do unsupervised training or train based on incomplete audiograms or a mix of incomplete and complete audiograms.

It is noted that the partial variational autoencoder is especially advantageous in enabling that the required training can be carried out based solely on incomplete audiograms or based on a mix of incomplete and complete audiograms.

This is advantageous for at least two reasons. One is that it increases significantly the amount of available training data, since all available audiogram are not measured based on common standard, as one example some measure hearing thresholds at seven frequencies for each ear while other use eight.

The other is that the inventors have realized that training (at least partly) with incomplete audiograms improves the ability of the partial variational autoencoder to subsequently predict based on incomplete audiograms. Hereby the autoencoder will require less time and less data (including measured hearing thresholds) to provide a precise prediction, which again will translate to a faster and hereby less cumbersome audiogram acquisition which especially will be advantageous for automated audiograms acquisition for e.g. fitting of Over-the-counter (OTC) hearing aids.

1. A hearing estimation system comprising an electro-acoustical transducer, a graphical user interface and processing means adapted to carry out the steps of:
a) using a plurality of audiograms and associated meta data to learn a latent representation wherein said plurality of audiograms and associated meta are provided from a plurality of hearing impaired persons wherein each of said hearing impaired persons has provided at least one of:
a complete or incomplete audiogram, and
at least one associated meta data,
and wherein said processing means is further adapted to carry out the steps of:
b) using said latent representation to provide a predictive distribution of a complete audiogram for a specific person, based on at least one of:
an incomplete audiogram of said specific person, and
at least one meta data of said specific person.

2. The hearing estimation system according to claim 1,
wherein step a) comprises the further step of:
training a variational autoencoder to provide said latent representation.

3. The hearing estimation system according to claim 2, wherein said step of training the variational autoencoder is carried out based on incomplete audiogram data only or based on both incomplete and complete audiogram data.

4. The hearing estimation system according to claim 2, wherein said variational autoencoder is a partial variational autoencoder.

5. The hearing estimation system according to claim 1, wherein step a) is carried out using unsupervised learning to learn the latent representation of an autoencoder.

6. The hearing estimation system according to claim 2, wherein said training of the variational autoencoder is carried out by optimizing a lower bound Lₚ on the log-marginal likelihood on the observed data xₒ:
$$Log {p}_{θ} ( {x}_{o} ) \geq {L}_{p} = {E}_{z ∼ {q}_{φ} ( z \vert {x}_{o} )} [ {\sum }_{o ∈ O} Log {p}_{θ} ( {x}_{o} \vert z ) - {D}_{KL} ( {q}_{φ} ( z \vert {x}_{o} ) \vert \vert p ( z ) ) ] = - ( {D}^{p} + R )$$
wherein D^{p}, denotes the expectation with respect to the approximate posterior E_{z˜q}_{_{φ}} (z|xₒ) of the negative log-likelihood of the observed data −Log p_{θ} (xₒ|z),
wherein R denotes the expectation with respect to the approximate posterior E_{z˜q}_{_{φ}} (z|xₒ) of the Kullback-Leibler divergence D_{KL} (q_{φ} (z|xₒ)∥p(z)) from the prior p(z), wherein the prior p(z) is a multi-variate normal distribution of the latent variable z, and
wherein xₒ is a vector comprising at least one of an observed frequency dependent hearing threshold and a meta data for said specific person, and
wherein xₒ represents an observed dimension of xₒ.

7. The hearing estimation system according to claim 1, wherein step b) comprises the further step of acquiring for said specific person at least one of:
at least one frequency dependent hearing threshold, and
at least one meta data,
and wherein step b) further comprises for said specific person the step of at least one of:
estimating a complete audiogram based on an average of the predictive distribution, and
selecting the next frequency for which to measure a frequency dependent hearing threshold; and
selecting the next sound pressure level to use for initiating the determination of a frequency dependent hearing threshold, and
estimating the uncertainty of an estimated complete audiogram, and
determining when to stop acquiring more frequency dependent hearing thresholds.

8. The hearing estimation system according to claim 7, wherein the step of estimating a complete audiogram based on an average of the predictive distribution comprises the steps of determining the predictive distribution p(x|xₒ) as:
$$p ( x \vert {x}_{o} ) = \int p ( {x}_{u} \vert z ) p ( {x}_{o} \vert z ) p ( z ) d ( z )$$
and determining the average μₓ of the predictive distribution as:
$${μ}_{x} = {E}_{p ( x \vert {x}_{o} )} [ x ] = \int x p ( x \vert {x}_{o} ) dx$$
and using μₓ as an estimate of the complete audiogram,
wherein x represents the complete audiogram, wherein z is the latent variable, wherein xᵤ represents unobserved frequency dependent hearing thresholds and wherein xₒ represents observed frequency dependent hearing thresholds.

9. The hearing estimation system according to claim 7, wherein the step of selecting the next frequency for which to measure a frequency dependent hearing threshold comprises the further step of using an acquisition function R(u, xₒ) based on the equation:
$$i = {arg max}_{u ∈ U} R ( u , {x}_{o} ) = {arg max}_{u ∈ U} {E}_{z ∼ {q}_{φ} ( z \vert {x}_{o} )} [ Var ( {p}_{θ} ( {x}_{u} \vert z ) ) ]$$
wherein i represents the next frequency to select,
wherein E_{z˜q}_{_{φ}} (z|xₒ) is the expectation with respect to the approximate posterior of the variance of the posterior predictive of the unobserved dimensions p_{θ} (xᵤ|z)
wherein Var(p_{θ} (xᵤ|z)) is approximated using the sample variance of samples from the posterior predictive of the unobserved dimensions p_{θ} (xᵤ|z) given multiple samples from the approximate posterior q_{φ} (z|xₒ).

10. The hearing estimation system according to claim 7, wherein the step of estimating the uncertainty Q of an estimated complete audiogram is given by:
$$Q = {\sum }_{i = 1}^{M} Var ( {x}_{i} ) = {\sum }_{i = 1}^{M} {[ Var ( x ) ]}_{i} = {\sum }_{i ∈ U} {E}_{z ∼ {q}_{φ} ( z \vert {x}_{o} )} ( {p}_{θ} ( {x}_{u} \vert z ) )$$
wherein M is the total number of frequencies to be measured in order to obtain a complete audiogram and,
wherein E_{z˜q}_{_{φ}} (z|xₒ) is the expectation with respect to the approximate posterior of the variance of the posterior predictive of the unobserved dimensions p_{θ} (xᵤ|z).

11. The hearing estimation system according to claim 7, wherein the step of determining when to stop acquiring more frequency dependent hearing thresholds comprises the step of:
detecting when an estimated uncertainty of an estimated complete audiogram drops below an uncertainty threshold.

12. The hearing estimation system according to claim 7, wherein the step of determining when to stop acquiring more frequency dependent hearing thresholds comprises the steps of:
using a model to predict when to stop acquiring more frequency dependent hearing thresholds, wherein said model is a neural network, a linear model or an un-linear model and wherein said model has been trained using ground truth data based from real audiogram acquisitions, and
wherein the input to the model at least comprises the number of measured frequency dependent hearing thresholds and an estimated uncertainty of an estimated complete audiogram.

13. The hearing estimation system according to claim 2, comprising the further steps of training at least one additional autoencoder for optimized performance with respect to one of:
estimating a complete audiogram, and
selecting the next frequency for which to measure a frequency dependent hearing threshold; and
selecting the next sound pressure level to use for initiating the determination of a frequency dependent hearing threshold, and
estimating the uncertainty of an estimated complete audiogram, and
determining when to stop acquiring more frequency dependent hearing thresholds.

14. A non-transitory computer readable medium carrying instructions which, when executed by a computer, cause the following method to be performed, the method comprising the step of:
using a latent representation of a plurality of at least one of audiograms and associated meta data from a plurality of hearing impaired persons to provide a predictive distribution of a complete audiogram for a specific person, based on at least one of:
an incomplete audiogram of said specific person, and
at least one meta data of said specific person.

15. A non-transitory computer readable medium carrying instructions which, when executed by a computer, cause the following method to be performed, the method comprising the step of:
using a latent representation of a plurality of at least one of audiograms and associated meta data from a plurality of hearing impaired persons to provide a predictive distribution of a complete audiogram for a specific person, based on at least one of:
an incomplete audiogram of said specific person, and
at least one meta data of said specific person; and
the method steps according to claim 7.

16. A method of training an algorithm for predicting a complete audiogram for a specific user, the method comprising:
providing a first database comprising, for each of a plurality of hearing impaired persons a vector comprising data representing at least one of an observed frequency dependent hearing threshold and a meta data;
training a deep neural network, in the form of a variational autoencoder, with at least some of said plurality of vectors to learn a latent representation of the data comprised in said plurality of vectors; by:
minimizing the distortions introduced by the composition of the encoder and decoder function of the variational autoencoder under a constraint on the rate of information passed through the latent space; or by:
optimizing a lower bound Lp on the log-marginal likelihood on the observed data; and
predicting a complete audiogram from an average of a predictive distribution based on said latent representation.
